(19) United States
(12) Patent Application Publication
(10) Pub. No.: US 2018/0322942 A1
(43) Pub. Date: Nov. 8, 2018

(54) MEDICAL PROTOCOL EVALUATION

(71) Applicant: 3M INNOVATIVE PROPERTIES COMPANY, St. Paul, MN (US)

(72) Inventors: Brian J. Stankiewicz, Mahtomedi, MN (US); Jennifer F. Schumacher, Woodbury, MN (US); Brian E. Brooks, St. Paul, MN (US); Nicholas A. Asendorf, St. Paul, MN (US); Kelly S. Peterson, Salt Lake City, UT (US)

(21) Appl. No.: 15/773,233

(22) PCT Filed: Oct. 27, 2016
(86) PCT No.: PCT/US16/58980

§ 371 Date: May 3, 2018

Related U.S. Application Data
(60) Provisional application No. 62/251,963, filed on Nov. 6, 2015.

(51) Int. Cl. G16H 10/60; G06N 99/00

(52) U.S. Cl. CPC G16H 10/60; G06F 17/28; G06N 99/005

(57) ABSTRACT

A computer-implemented method of evaluating a plurality of protocols associated with a medical context includes receiving, with a computer system, an indication of a medical context item corresponding to a medical context, accessing, with the computer system, a digital library including a plurality of protocols associated with the medical context, assigning, with the computer system, predictive outcomes to one or more of plurality of protocols, selecting, with the computer system, one of the plurality of protocols associated with the medical context based upon the assigned predictive outcomes, and storing, with the computer system within a database, an indication the selected protocol is assigned to the medical context item.

### TECHNICAL FIELD

This disclosure relates to computer-based analysis of medical records.

### BACKGROUND

Everyday thousands of medical facilities use a multitude of different protocols to conform to standards of care within the facility. These protocols may be developed according to many different sources and are generally defined as the description of steps taken to provide care and treatment to one or more patients or to provide safe facilities and equipment for the care and treatment of patients. Protocols may include, for example, a list of recommended steps, who performs aspects of the steps, and where the steps should be performed. In- and out-patient medical facilities may adopt, and/or modify, their protocols from research papers, professional journals, or public knowledge that describes and provides suggestions for the best practices. Additionally, protocols can be created by observation and intuition made by facility personnel and staff. Such protocols may be developed over time and may change in response to additional information, such as adverse events, medical studies and additional input from medical facility personnel and staff.

### SUMMARY

This disclosure is directed to computer-based techniques for evaluating a plurality of protocols associated with a medical context. As referenced herein, a medical context defines a set of items, such as events or circumstances, related to the care, operations and/or treatment of one or more patients and/or the medical environment. Each unique and/or individual circumstance meeting the definition of a medical context is referred to herein as a medical context item. In some examples, a medical context may be defined according to a patient condition, and may optionally include further patient history or other patient attributes; in other examples, a medical context may represent other circumstances not directly associated with a patient in which a medical protocol is applied, such as room or equipment cleaning procedures or other facilities and equipment management practices. A single medical context item, such as the condition and other attributes of a single patient, may be categorized with other related items that also meet the definition of a medical context. The medical context defines some attributes of related items to facilitate analysis of medical protocols applied to the medical context items.

In various examples, the disclosed techniques may be used to evaluate a plurality of protocols associated with a medical context. In the same or different examples, the techniques described within may be used to identify a subset of the medical context items (e.g., 40-something males who are newly diagnosed diabetic) for which none of the evaluated protocols have a significant impact or efficacy. Such contexts are referred to as low-efficacy medical contexts, or more specifically with respect to a patient population, as a low-efficacy patient population.

In one example, this disclosure is directed to a computer-implemented method of evaluating a plurality of protocols associated with a medical context, the method comprising receiving, with a computer system, an indication of a medical context item corresponding to a medical context, accessing, with the computer system, a digital library including a plurality of protocols associated with the medical context, assigning, with the computer system, predictive outcomes to one or more of plurality of protocols, selecting, with the computer system, one of the plurality of protocols associated with the medical context based upon the assigned predictive outcomes, and storing, with the computer system within a database, an indication the selected protocol is assigned to the medical context item.

In another example, this disclosure is directed to a computer system-readable storage medium that stores computer system-executable instructions that, when executed, configure a computer system to perform the preceding method.

In another example, this disclosure is directed to a computer system comprising one or more processors configured to perform the preceding method.

In another example, this disclosure is directed to a computer-implemented method of evaluating a plurality of protocols associated with a medical context, the method comprising accessing, with a computer system, a database including medical information for a plurality of patients associated with medical context items corresponding to the medical context. For each of the plurality of patients, the medical information includes an indication that one of the plurality of patient protocols is associated with the patient. The method further includes evaluating, with the computer system, each of the plurality of patient protocols based on medical information associated with patients within a patient population, to estimate an efficacy of each of the plurality of patient protocols for the patient population, wherein the patient population represents a subset of the plurality of patients, identifying, with the computer system, the patient population represents a low-efficacy patient population based on the efficacy estimates for the patient population, and storing, within the database, an indication that the patient population represents the low-efficacy patient population.

In another example, this disclosure is directed to a computer system-readable storage medium that stores computer system-executable instructions that, when executed, configure a computer system to perform the preceding method.

In another example, this disclosure is directed to a computer system comprising one or more processors configured to perform the preceding method.

The details of one or more examples of this disclosure are set forth in the accompanying drawings and the description below. Other features, objects, and advantages associated with the examples may be apparent from the description and drawings, and from the claims.

### BRIEF DESCRIPTION OF DRAWINGS

FIG. 1 illustrates a network including computer system for searching and identifying medical context items within medical documents.

FIG. 2 is a diagram illustrating example applications of the network of FIG. 1 to manage evaluations of protocols associated with a medical context.

FIG. 3 is a flowchart illustrating example techniques for developing an evaluation of a plurality of protocols associated with a medical context.

FIG. 4 is a flowchart illustrating more detailed example techniques than those of FIG. 3 for developing an evaluation of a plurality of protocols associated with a medical context.

FIG. 5 is a flowchart illustrating example techniques for evaluating a plurality of protocols associated with a medical context.

FIG. 6 is a flowchart illustrating example techniques for identifying a patient population as a low-efficacy patient population based on efficacy estimates of a plurality of protocols for the patient population.

FIG. 7 is a block diagram of an example configuration of a computer system, which may be used for evaluating a plurality of protocols associated with a medical context.

### DETAILED DESCRIPTION

This disclosure is directed to computer-based techniques for evaluating protocol assignment and management based on medical contexts. In various examples, the disclosed techniques may be used to evaluate a plurality of protocols associated with a medical context. As referenced herein, a medical context defines a set of items, such as events or circumstances, related to the care, operations and/or treatment of one or more patients and/or the medical environment. In some examples, a medical context may represent a patient condition, and may optionally include further patient history or other patient attributes; in other examples, a medical context may represent other circumstances not directly associated with a patient in which a medical protocol is applied, such as room or equipment cleaning procedures or other facilities and equipment management practices. A single medical context item, such as the condition and other attributes of a single patient, may be categorized with other related items that also meet the definition of a medical context. Recording and analyzing defined performance metrics for medical context items over time to create and update a database facilitates predicting outcomes for different medical protocols applied to the medical context. In various examples, the disclosed techniques may be used to evaluate and compare a plurality of protocols associated with a medical context.

In the same or different examples, the techniques may be used to identify a patient population that represents a low-efficacy patient population based on efficacy estimates for a plurality of protocols applied to the patient population.

In addition to using the structured data that is available (e.g., age, gender, medical diagnosis codes, etc.) the techniques optionally include using natural language processing (NLP) for searching and identifying medical context items within medical documents. NLP techniques may allow users to analyze data and attain knowledge from electronic medical records and any other available documents that contain either free text (e.g., unstructured) components and/or structured components. Example NLP techniques that may be used include, and are not limited to: pattern matching, statistical machine learning, syntactic-driven parsing (i.e., decision trees), semantic grammar transformation, deep learning, phrase detection and the like.

There are many methods that may be used to define protocols within a medical facility. However, significant challenges are faced when collecting evidence to support the definition, evaluation and assignment of a protocol for a given medical context. Designing and running controlled or semi-controlled experiments to define and/or evaluate protocols can be difficult, resource intensive, and time consuming. Furthermore, many facilities do not have the expertise to conduct such studies to obtain the relevant information for evaluation.

Before evaluation of multiple protocols for a medical context can occur, the protocols themselves need to be selected for comparison and evaluation. The protocols may be assembled from the current protocol or protocols associated with an institution, such as a medical facility, care provider or insurance company, as well as additional protocols that are not associated with the institution, such as from peer-reviewed literature. Additional protocols may originate from other institutions, such as a different medical facility, care provider or insurance company. Once developed, identified and/or selected, such protocols may be stored in a protocol library. Additional protocols may be developed by modifications to protocols within the protocol library. For example, if, for a given set of protocols with the library, the best predictive outcomes are from a protocol with highest value of a quantitative factor (e.g., most patient reminders, highest dose of a drug, most frequent rehab appointments, etc.), then it may make sense to create and evaluate a new protocol with an even higher value for the quantitative factor, such as even more patient reminders, higher dosing of the drug, even more frequent rehab appointments, etc. In various examples, such modifications to other protocols within the protocol library may be automatically selected by a computer system, e.g., using a machine learning algorithm, and/or defined by a user.

Once a protocol library is assembled with the plurality of protocols in the form of a database for a given medical context, a computerized system selects and assigns protocols from the library to medical context items. The computerized system then monitors any of the performance metrics (e.g., length-of-stay, readmission, infection) associated with the protocol and the medical context items. The performance metrics are used to evaluate the protocols within the library. Through the techniques disclosed herein, the computerized system learns the expected impact that these protocols have on the performance measures (i.e., outcomes) for a given medical context item.

The computerized system may limit or bias the selection of protocols within the library to those in which the predicted impact (or performance measures) is highly uncertain or currently not predicted to be the best outcome, and among the protocols that have the best predicted impact for the medical context. Thus, a balance is enacted between the process of gathering new outcome information (explore) and leveraging that knowledge to improve the outcome (exploit). Machine learning techniques may be implemented to assist with the computerized data exploration and exploitation. Machine learning techniques that may be used include: reinforcement learning, Markov Modeling, naïve Bayesian classifiers, neural networks, symbolic learning, decision trees, and the like. As machine learning commences, the amount of exploration reduces and exploitation increases.

As discussed above and further described below, a user may create or select protocol content associated with a medical context, distribute the protocol content for medical context items to evaluate different protocols, and evaluate the results and ultimately improve protocols for specific conditions (e.g., hospital, patient, physician, etc.) associated with a medical context. The results may be used to update predictive outcomes for the protocols. Comparing the predicted outcomes among the evaluated protocols facilitate evaluating the relative effectiveness of protocols associated with a medical context. By future selection of highly-effective protocols, the outcomes for the medical context may be improved.

In addition, a single medical protocol may not provide an encompassing solution for the various medical context items meeting the definition of the medical context. For example, with respect to patient protocols, there may be factors beyond those defined by the medical context that make one protocol better suited for one context item over another context item. These factors might be based upon patient and facility demographics. Techniques disclosed herein further address this issue of identification of a subset of the medical context items (e.g., 40-something males who are newly diagnosed diabetic) for which none of the evaluated protocols have a significant impact or efficacy. Such medical contexts defined by the subset of evaluated medical context items are referred to as low-efficacy medical contexts, or more specifically with respect to a patient population, as a low-efficacy patient population. Using machine-learning techniques such as regression and active learning the system can identify these factors and medical context items that are low-efficacy. Once such low-efficacy medical contexts are identified, further protocols may be developed and evaluated to provide improved protocols for the low-efficacy medical contexts.

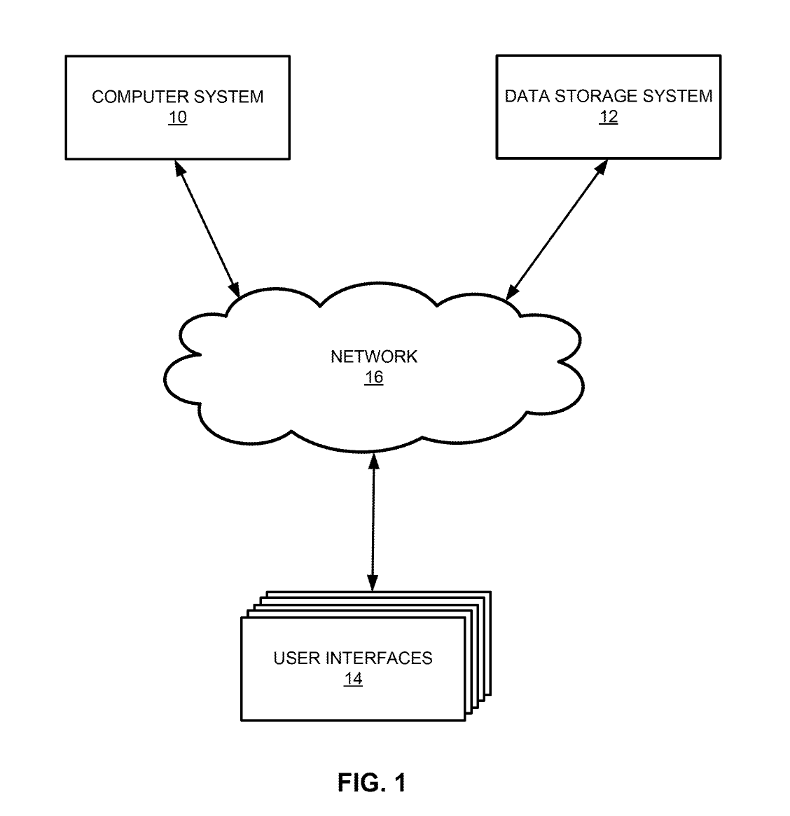

FIG. 1 illustrates a network computer system for evaluating a plurality of protocols associated with a medical context. The network shown in FIG. 1 includes computer system 10, data storage system 12, user interfaces 14 and network 16, which serves to communicatively couple each of computer system 10, data storage system 12 and user interfaces 14 to one another. In some examples, user interfaces 14 may be associated with a single institution, such as a medical facility, medical service provider, or an insurance company. In other examples, user interfaces 14 may be distributed across multiple institutions such that the techniques described herein may facilitate the evaluation of protocols for a medical context across multiple institutions. In other examples, a subject institution may compare its preferred protocols (performance and design) for a medical context with protocols from other institutions.

In different examples, network 16 may represent a computer bus, a local area network (LAN), a virtual private network (VPN), the Internet, a Cloud based network, or a combination thereof or any other network. For example, network 16 may comprise a proprietary on non-proprietary network for packet-based communication. In one example, network 16 comprises the Internet and data may be transferred via network 16 according to the transmission control protocol/internet protocol (TCP/IP) standard, or the like. More generally, however, network 16 may comprise any type of communication network, and may support wired communication, wireless communication, fiber optic communication, satellite communication, or any type of techniques for transferring data between a source (e.g., data storage system 12) and a destination (e.g., computer system 10).

In accordance with the techniques described herein, computer system 10, may optionally receive an indication of at least one medical context item via user interfaces 14. Computer system 10 may automatically identify medical contexts items meeting the definition of a medical context based on indirect indications of the medical context items in medical information, such as patient or medical facility information. In such examples, the indication of the medical context item is the medical information itself, rather than a direct indication from user of the presence of a medical context item. In either example, following the identification of a medical contact item, computer system 10 may access a protocol library including a plurality of protocols corresponding to the medical context of the medical context item, and may send instructions corresponding to the selected one of the plurality of protocols. Instructions may include a full or partial description of the protocol along with steps or procedures that a patient or other person (e.g., medical facility or institution personnel such as nurses, physicians, etc.) should follow to support the selected protocol to improve patient care and/or treatment. Computer system 10 may further monitor medical information associated with the medical context item following the selection of the one of the plurality of protocols. For example, computer system 10 may monitor patient outcomes associated with the medical context item.

Computer system 10 may evaluate each of the selected protocol based on performance measures associated the medical context items. Performance measures may be quantitative (e.g., a scalar, a range, etc.) or qualitative (e.g., industry or facility defined descriptions). Example quantitative performance measures include: length of stay, compliance rates, admissions, readmissions, discharges, occupancy, infection rates, inpatient or outpatient days, or the like. Examples of qualitative performance measures include: quality of care, reports of communication, facility appearance and cleanliness, or the like.

In some examples, computer system 10 may update efficacy estimates for the selected protocol, and store the updated efficacy estimates for the selected protocol within a database. By applying such techniques across multiple medical context items using different protocols in the plurality of protocols, computer system 10 may build a database providing reliable efficacy estimates for each of the plurality of protocols associated with a medical context.

In some examples, computer system 10 may access data storage system 12 to retrieve all or a portion of the medical context items, to retrieve predetermined ontologies and/or quantitative factors associated with the medical context and/or store updates to efficacy estimates for each of the plurality of protocols. As referred to herein, an indication of a medical context may be a label for the medical context, such as a word, phrase, acronym, abbreviation, or other label for the medical context. An indication of a medical context may represent an ontology of a selected indication of the medical context or quantitative factors associated with the medical context. In this manner, computer system 10 may determine medical context items according to specified criterion without each medical context item needing to be precisely labeled according to the criterion.

Medical protocols refer to a prescribed series of actions designed to improve patient or facility outcome defined by a medical context. Each unique and/or individual circumstance defined by a medical context is referred to herein as a medical context item. Protocols associated with a medical context can be formulated at the hospital-level (e.g., a protocol with cleaning instructions to reduce hospital acquired infections), caretaker-level (e.g., a protocol for hand cleaning), and/or the patient-level (e.g., a protocol to ensure that a patient fulfills their prescription). For a given medical context, protocols may or may not exist for a given facility.

In some examples, a medical context may represent a patient context, such as any attribute or combination of attributes associated with a patient. Such attributes include, but are not limited to, a chief complaint of the patient, a history of present illness of the patient, a past medical history of the patient, a social history of the patient, a family history of the patient, a review of systems of the patient, allergies of the patient, medications of the patient, impressions of the patient by a clinician, a medical plan for the patient, diagnostic imaging results performed on the patient, results of a medical test of the patient, a gender of the patient, an ethnicity of the patient, an age of the patient, a physical attribute of the patient, physical signs of the patient, physical systems of the patient, a time period associated with one of the preceding attributes or another attribute, and/or other attributes. A medical context may be associated with patients associated with a selected attribute, not associated with a selected attribute, and/or associated with patients for which the selected attribute is unknown.

In some examples, a medical context item may be detected by computer system 10 according to medical documents. Such medical documents may include any of the following categories of medical documents: government-acquired medical documents from a Medicare repository, medical documents submitted to a government by the medical facility, medical documents submitted to the government by many medical facilities, medical documents received from one or more medical facilities, medical documents received from one or more insurance companies, medical documents associated with all-payer health insurance claims, and other medical documents. As referred to herein, medical facilities include hospitals, clinics, laboratories performing analysis or medical testing, and other facilities associated with the treatment or diagnosis of medical patients.

In the same or different examples, the medical documents may include electronic medical records (EMR) or electronic health records (EHR), medical clinician notes, medical clinician dictations, medication files, radiology reports, emergency department reports, patient pathology reports, and other medical documents. In more specific examples, the medical documents may include documents associated with one or more of the following: allied services—occupational therapy, allied services—physical therapy, emergency department—nursing, emergency department—physician, emergency department—triage, inpatient—admission nursing note, inpatient—admission physician history and physical, inpatient—discharge instructions, inpatient—discharge summary, inpatient—nursing progress, inpatient—physician discharge summary, inpatient—physician orders, inpatient—physician progress, medical specialty—cardiology, medical specialty—endocrinology, medical specialty—gastroenterology, medical specialty—pulmonology, medical specialty—radiology, operative procedures, outpatient—nursing progress notes, outpatient—physician progress notes, pathology—anatomic, pathology—laboratory, surgery specialty—cardiac surgery, surgery specialty—obstetrics and gynecology, surgery specialty—orthopedic surgery and other documents. The medical documents listed and described herein are merely examples. Computer system 10 may automatically detect a medical context item associated with a protocol in order apply the techniques disclosed herein with respect to evaluation of a medical protocol. In other examples, a user may indicate to computer system 10 when a medical context item associated with a medical protocol exists.

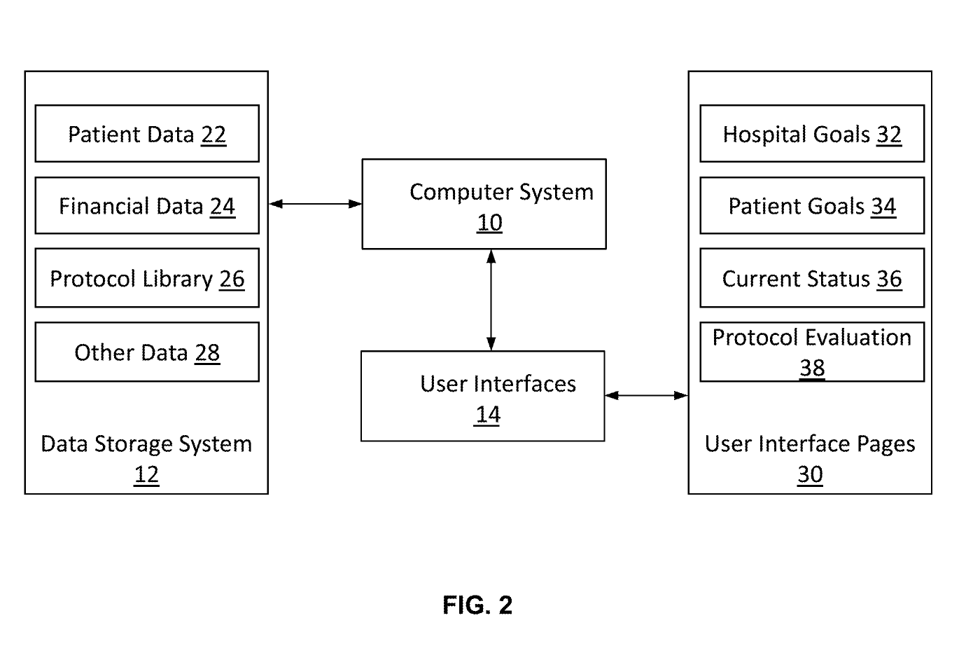

FIG. 2 is a diagram illustrating example applications of the network of FIG. 1 to manage evaluations of protocols associated with a medical context. FIG. 2 includes data storage system 12, user interfaces 14 and user interface pages 30. User interface pages 30 facilitate techniques for manually and automatically creating, updating, evaluating and selecting protocols. To facilitate the creation and selection of the one or more protocols, a user may define the parameters or variables (e.g., independent variables) that can influence performance through the user interface pages 30. Generally speaking, the independent variables define the medical context that leads to the selection of the appropriate protocol. For example, at the hospital-level, a user may be interested in selecting a protocol for a room (environment) in which a previous patient had sepsis.

The user may also define measured variables or performance metrics that are indicators of the effectiveness of the protocol for a given medical context. For example, the user may measure the rate of hospital-acquired infections (outcome measure) and analyze the count of bacteria colonies on specific surfaces within a room (indicator measure). In this specific example, a user may use Clean Trace products available from 3M Company of St. Paul, Minn., to measure this directly. In some examples, the measured variables would be values that would be constantly measured within the hospital regardless of the protocol. In some examples, the statistical variability of the measured variables would be available or calculated and may be used to automatically provide insight as to what protocols would be effective to measure significant results. Using this insight, the user may modify the evaluation by reducing the complexity of the protocol selection (e.g., identify fewer protocols) if the user thought that it would take too long or add additional evaluations if the value of the evaluation justified the time.

As shown in FIG. 2, the network may integrate data from a variety of sources, including, but not limited to, patient data 22, financial data 24, protocol library 26, and other data 28. Patient data 22 may include data indicating a patient medical context and/or patient outcome data. Other data 28 may include information specific to a medical facility or operations of the medical facility rather than medical information directly associated with patients, such as average length-of-patient stay, occupancy rates, available emergency services, admissions, discharges, infection rates, room/facility condition, etc. Financial data 24 may include cost information for medical therapies and facilities, and/or cost information specifically associated with the treatment of patients. In examples in which financial data 24 includes cost information for medical therapies and facilities, the cost information associated with patient outcomes in patient data 22 may be estimated or calculated based on financial data 24. Protocol library 26 may include protocol descriptions such as procedures and patient communication methods or simply a listing of protocol identifiers, which may or may not include actual information describing protocols.

A user interacts with the network via user interfaces 14. User interface 14 may include a display screen for presenting visual information to a user. In some embodiments, the display screen includes a touch sensitive display. In some embodiments, user interface 14 may include one or more different types of devices for presenting information to a user. FIG. 2 illustrates example user interface pages 30. The user interface pages 30 of FIG. 2 are shown as one example, but those of skill in the art would appreciate that other examples may be consistent with the present disclosure. The illustrated user interface pages 30 are shown merely to explain various aspects of the present disclosure and the addition or removal of components would be apparent to one of skill in the art. As one example, a user may interact with the network via user interface page 32, which lists “Hospital Goals” or by interface page 34, which lists “Patient Goals.” By selecting goals, a user may be presented with potential medical contexts that could be evaluated using the techniques described herein. The user may select both the criteria for evaluation or “goal,” the relative improvement, or reduction of one of the core measures. Hospital and/or patient goals (32, 34) may be presented to the user as checklists, drop-down menus, text boxes, or the like. Goals may be predefined and loaded within the user interface or may be defined by the user in a current protocol evaluation session. As an example, a hospital goal 32 may be defined as identifying the most effective protocols to manage 40-something males who were newly diagnosed as diabetic. The patient goal 34 may be defined as how to manage newly diagnosed diabetes with increased exercise and minor modification to diet.

Interface page 36 provides a “Current Status,” of an evaluation, such as summary statistics of the current status of goals being evaluated. Current status may be presented visually through graphical charts, bar graphs, histograms, textual summaries or the like. Similarly, interface page 38 provides “Protocol Evaluation,” which may include displaying the protocols and their metrics as well as tools to initiate further evaluation of protocols. Protocol evaluations may be presented as summaries in report or other textual formats.

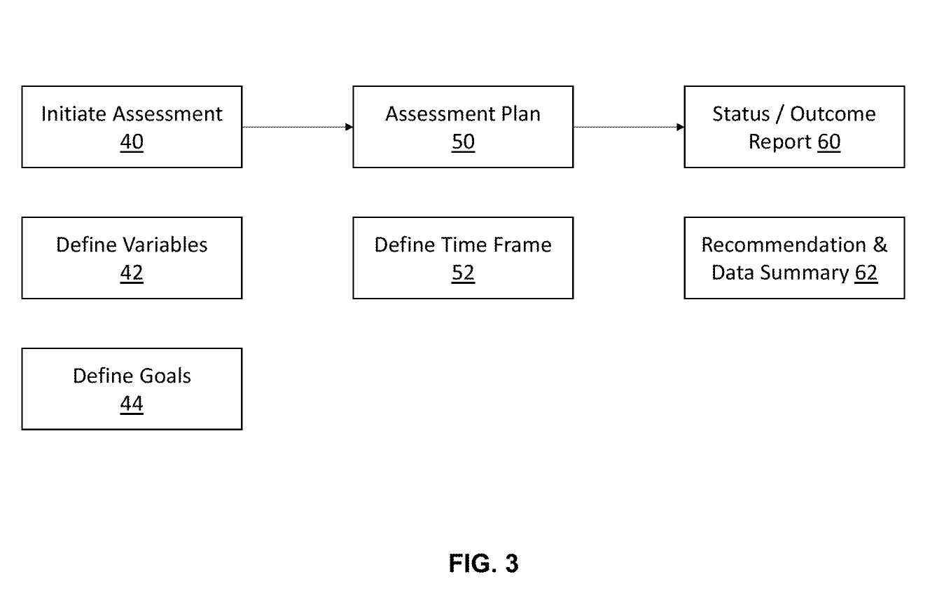

FIG. 3 is a flowchart illustrating example techniques for developing an evaluation of a plurality of protocols associated with a medical context using computer system 10 (FIG. 2). As shown in FIG. 3, the first step is to initiate a protocol assessment 40. Initiating the protocol assessment includes defining variables 42 (e.g., medical context items or medical context) as well as the goals 44 (i.e., evaluation metrics). These techniques may utilize the user-defined parameters to define the evaluation of the protocols. These protocols could be defined at the level of a facility (e.g., hospital), ward (e.g., a floor within a hospital), caretaker (e.g., nurse) or be specific to a patient.

Once a user defines the variables and goals, computer system 10 defines the evaluation specificity according to assessment plan 50. The specificity is dependent on a number of incidences, such as time frame and repetitions 52, as well as variability of the results, which may be unknown at the initiation of the evaluation. In order to implement the evaluation, computer system 10 automatically assigns different protocols to each medical context within the evaluation for specific periods of time. At the end of the assessment, computer system 10 can provide a status and outcome report 60, which may include recommendations for improving the protocol(s) and/or a summary of the results 62 for analysis and conclusion.

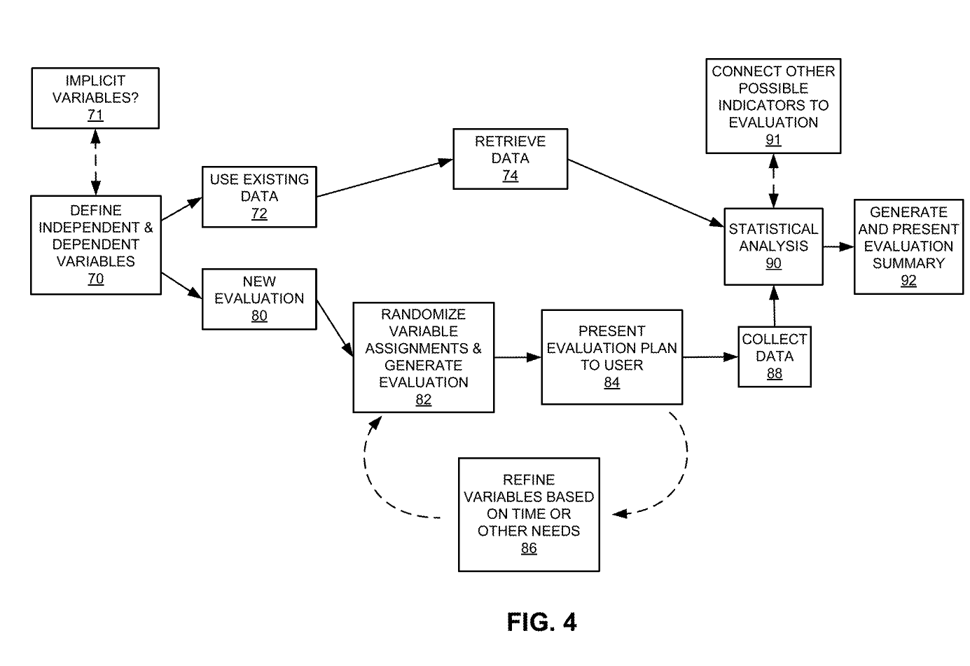

FIG. 4 is a flowchart illustrating more detailed example techniques than those of FIG. 3 for developing and evaluating one or more of protocols associated with a medical context. First, a user may define independent and dependent variables (70). Independent variables define a medical context, such as a patient who is newly diagnosed as diabetic, whereas dependent variables are used to evaluate the medical context, such as the patient is male and 42 years old. Optionally, computer system 10 further includes implicit variables (71) based on the user selected independent and dependent variables (70). For example, in a medical setting, implicit variables may include the facility in which a protocol is being applied, the use of sterilized equipment, and/or a vendor of the equipment used during the treatment of the patient.

Computer system 10 then accesses existing data (72) in data storage system 12 and determines whether the existing data is sufficient for protocol evaluation, in which case computer system 10 retrieves the data (74), and computer system 10 performs statistical analysis (90) on the existing data. The statistical analysis includes assignment of a predictive outcome for the protocol. The predictive outcome may calculated and presented as a percentage, score, efficacy rating, or the like. For example, computer system 10 may evaluate dependent variables for each protocol to determine the effectiveness of the protocol using a machine learning algorithm, such as ε-Greedy, Greedy, or other machine learning algorithms, based on: 1) prior performance of the plurality of protocols in the medical context items, 2) an expected performance of the one protocol from the plurality of protocols, 3) a counter-balanced assignment of contexts to protocols, 4) maximizing information expected to be obtained by the selection, and/or 5) other factors and techniques.

When retrieving data (74) computer system 10 may search medical documents for medical context items and results using NLP. Such techniques may provide significantly more information than using only formally labeled and sorted data. Within a set of medical documents, while clinicians tend to utilize a standardized approach for annotating a patient encounter, how the document is dictated, including how the sections are labeled, the order of the sections, whether or not section titles exist and, if so, whether the sections are explicitly marked, varies tremendously between different institutions and between doctors at the same institution. Indeed, an individual doctor's dictation patterns may vary, either based upon the type of exam or procedure they are performing, or for completely arbitrary reasons. An NLP engine may perform a regioning analysis on each document to map the variation to the standard note types and normalized region titles listed above.

Optionally, computer system 10 may index data parsed from the medical documents to facilitate parsing for corresponding indications of medical context items. In addition, the computer system may retrieve the medical documents from memory or from a data storage system, such as data storage system 12 (FIG. 1). Optionally, computer system 10 may acquire the medical documents by receiving the medical documents and/or an indication of location(s) of the medical documents via a network connection.

In some examples, computer system 10 may access a database or library identifying ontologies of the indication of the medical context items received by computer system 10 and/or identifying quantitative indications of the medical context. In other examples, the indications that correlate to the indication of the medical context received by the computer system may include quantitative indications of the medical context. For example, if a medical context is defined by hypertension, quantitative indications of a medical context may include blood pressures above a defined range for a patient. In examples where the indications that correlate to the indication of the medical context received by the computer system may include quantitative indications of the medical context, computer system 10 may access a database identifying the quantitative indications of the medical context.

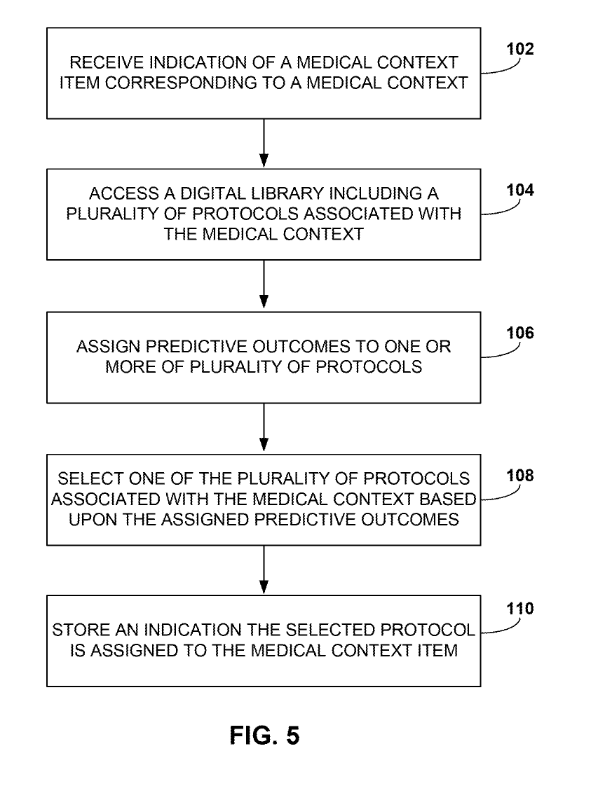

Alternatively or in addition to performing statistical analysis on the existing data, computer system 10 may begin a new evaluation (80) by designing and creating techniques to collect additional data for statistical analysis. In such examples, computer system 10 selects protocols from a plurality of protocols for each medical context item. The protocols may be randomly selected or selected according to other techniques, such as ε-Greedy or Greedy as described below with respect to FIG. 5. Computer system 10 further generates an evaluation plan for the different selected protocols (82). Computer system 10 optionally presents the evaluation plan for the different selected protocols to a user (84). The user may optionally refine the variables selected in step 70 based on time, repetition, or expected results indicated by the evaluation plan (86). Then, computer system 10 monitors information relating to each medical context item to collect data for the evaluation (88). The collected data may be optionally combined with preexisting data, and computer system 10 performs statistical analysis (90) on the collected data, and optionally on existing data.

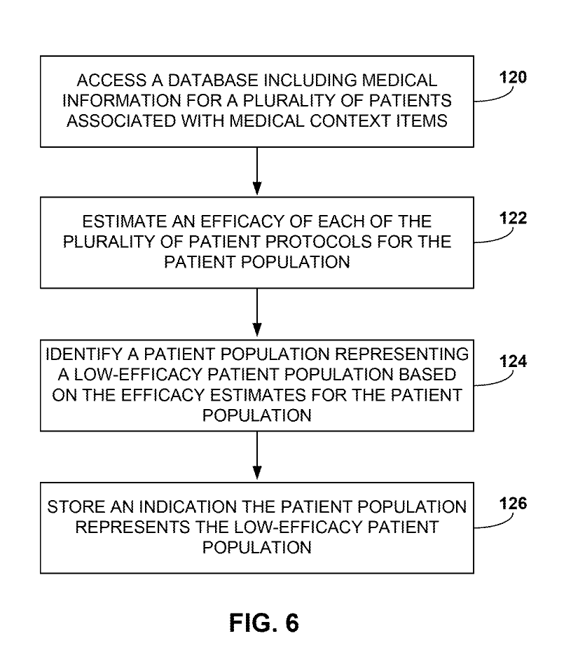

Once computer system 10 has performed the statistical analysis, computer system 10 may optionally connect additional independent variables (indicators) to the evaluation (91). For example, computer system 10 may identify a low-efficacy patient population as described in further detail with respect to FIG. 6. In any event, once computer system 10 has performed the statistical analysis, computer system 10 generates and presents an evaluation summary for the plurality of protocols to a user (92).

In an example application of the techniques of FIG. 4, a medical facility may evaluate the effectiveness of different room-cleaning protocols in which they have three different cleaning solutions (Solution-A, Solution-B and Solution-C) and two different procedures (Spray & Wipe, Spray & Dry) for a room that previously contained a patient with sepsis. Such an evaluation provides that there would be six different protocols (Sol.-A-Wipe, Sol.-A-Dry, Sol.-B-Wipe, Sol.-B-Dry, Sol.-C-Wipe and Sol.-C-Dry). Given these six protocols, the measured variables may include “Colony-Count-Bedrail”, “Colony-Count-Door handle”, “Colony-Count-Cupboard”, and “Infection-Rate.” A user may determine if data already exists within data storage system 12 (72) or may define a new evaluation protocol (80). Either way, analysis would be performed (90) based on the measured variables to select which protocol would be the most effective to clean the room.

Computer system 10 may also identify other “implicit” variables for the evaluation (71), such as the hospital type or the physician's training history that could be used for further improvement and/or the identification of future studies. If the evaluation protocol already has sufficient data (72), that data would be extracted from data storage system 12 (74), analyzed (90), and presented to the user (92). If defining a new evaluation protocol (80), computer system 10 would randomize the conditions and assign them to different hospitals/physicians/cleaning teams (82) and propose an evaluation plan to the user (84). If the user would like to edit the protocol based on time, repetition, or other needs, the user would be presented the option (86) and computer system 10 could update the evaluation plan (82). Data would then be collected (88), and other possible indicators (91) would be connected during the statistical analysis (90). These indicators may not be directly associated with the defined measured variables, but they may help predict the outcome or play a causal role. Finally, the results would be generated and presented to the user (92). This could include, but is not limited to, suggesting protocol changes based on relative probabilities of the impact of other variables (e.g., suggest using a type of cleaning solution as a variable rather than the person doing the cleaning). The method of communicating the protocol evaluation results could vary depending on the level of analysis or could even be tailored for each user's preference or known method of preferred follow-through (e.g., email results and reminders to user A, send daily text messages to user B, etc.).

FIG. 5 is a flowchart illustrating example techniques for evaluating a plurality of protocols associated with a medical context. More specifically, the techniques of FIG. 5 facilitate comparing and evaluating a protocol for a medical context item meeting the definition of a studied medical context.

As referenced herein, a medical context defines a set of items, such as events or circumstances, related to the related to the care, operations and/or treatment of one or more patients and/or the medical environment. In some examples, a medical context may represent a patient condition, and may optionally include further patient history or other patient attributes; in other examples, a medical context may represent other circumstances not directly associated with a patient in which a medical protocol is applied, such as room or equipment cleaning procedures or other facilities and equipment management practices. In addition to the numerous other examples disclosed herein, a medical context may be defined according to one or more of: a chief complaint of a patient, a history of present illness of the patient, a past medical history of the patient, a social history of the patient, a family history of the patient, a review of systems of the patient, allergies of the patient, medications of the patient, impressions of the patient by a clinician, a medical plan for the patient, diagnostic imaging results performed on the patient, results of a medical test of the patient, a gender of the patient, an ethnicity of the patient, an age of the patient, a physical attribute of the patient, physical signs of the patient, and physical systems of the patient.

As shown in FIG. 5, the techniques include receiving, with a computer system, such as computer system 10 (FIG. 1), a database, such as data storage system 12, an indication of a medical context item corresponding to the medical context (102). Computer system 10 may identify patients as being associated with the medical context based on patients who are associated with medical documents that include the indication of the medical context, such as identifying patients in the plurality of patients as being associated with the medical context based on quantitative indications of the medical context within the medical documents. In the same or different examples, computer system 10 may receive, from one or more users via one or more user interfaces, an indication that a patient is associated with the medical context item.

In some examples, for each of the medical context items, computer system 10 may access a digital library including the plurality of protocols associated with the medical context items (104). In some examples, computer system 10 may be used to populate such a digital library with one or more of the plurality of protocols associated with the medical context item. For example, computer system 10 may interrogate a user regarding preferred protocols at their institution and/or request additional protocols the user would like to evaluate such that protocols will be associated with the institution of the medical context items being used in the evaluation. In addition, computer system 10 may suggest additional protocols, including protocols associated with an institution not associated with at least some of the medical context items and/or protocols based on peer-reviewed literature. Generally, a user will have the option of accepting, rejecting or modifying protocols suggested by computer system 10.

Computer system 10 assigns predictive outcomes to one or more of plurality of protocols associated with the medical context. For example, the assigned predictive outcomes may be calculated based on medical information associated with medical context items, random selection of the protocols, the effectiveness of how the protocol addressed a previously analyzed medical context, the total number of protocols available for selection in the library, and/or the total number of protocols specific to a medical context in the library.

Computer system 10 may select one of the plurality of protocols associated with the medical context based upon the assigned predictive outcomes (108). For example, computer system 10 may select one of the plurality of protocols associated with the medical context based upon a random selection of protocols meeting on or more criteria, such as a minimal level of predicted effectiveness, counter-balanced assignment of medical contexts to protocols, maximizing information expected to be obtained by the selection, according to a machine learning algorithm and/or according to other techniques.

For some of the plurality of patients, the medical information may include an indication of one of the plurality of patient protocols associated with the patient. For example, the patient may have been associated with the patient protocol prior to the initialization of the evaluation of the plurality of patient protocols or after the initialization of the evaluation of the plurality of patient protocols. For other medical context items (e.g., patients), computer system 10 selects one of the plurality of protocols associated with the medical context based upon the assigned predictive outcomes (108). For example, computer system 10 may select the protocol at random, based on: 1) a machine learning algorithm, such as ε-Greedy, Greedy or other machine learning algorithm, 2) prior performance of the plurality of protocols in the medical context items, 3) an expected performance of the one protocol from the plurality of protocols, 4) a counter-balanced assignment of contexts to protocols, 5) maximizing information expected to be obtained by the selection, and/or 6) other factors and techniques. Following the selection of the medical context, for each of the medical context items, computer system 10 stores an indication the selected protocol is assigned to the medical context item within a database (110).

Computer system 10 may also send instructions corresponding to the selected protocol. The instructions may include a full or partial description of the selected protocol or only a protocol identifier without actual information regarding the protocol itself.

Computer system 10 may also monitor medical information associated with the medical context item following the selection of the one of the plurality of protocols. For example, monitoring medical information may include monitoring patient information, such as patient information of a patient corresponding to one of the medical context items. Such medical information may include indications of patient readmissions among the plurality of patients, indications of medical expenses among the plurality of patients, indications of medical outcomes among the plurality of patients, a characterization of compliance with selected protocols among the plurality of patients such as indications that a non-selected one of the plurality of protocols was applied to one or more of the plurality of patients.

Computer system 10 may further evaluate the selected protocol based on performance measures associated with the medical context item to update efficacy estimates for the selected protocol. Computer system 10 may store the updated efficacy estimates for each of the plurality of protocols within a database, such as data storage system 12.

The technique of FIG. 5 may be repeated for a plurality of medical context items corresponding to a medical context in order to provide updated predictive outcomes for each of the plurality of protocols associated with a medical context. In this manner, the techniques of FIG. 5 may be used to iteratively learn and compare and contrast predictive outcomes of a plurality of protocols associated with a medical context.

In some examples, computer system 10 may also evaluate each of the plurality of protocols based on the medical information associated with one or more patient groups representing a subset of the plurality of patients to update an efficacy of the each of the plurality of protocols for the one or more patient groups. As discussed in further detail with respect to FIG. 6, such techniques may be used to identify one or more low-efficacy patient groups based on the efficacy estimate among patients within the plurality of patients and within the one or more low-efficacy patient groups.

FIG. 6 is a flowchart illustrating example techniques for identifying a patient population as a low-efficacy patient population based on the efficacy estimates for the patient population. The techniques of FIG. 6 may be combined with the techniques of FIG. 5 or may be independent from the techniques of FIG. 5.

During the protocol evaluation and data collection techniques described with respect to FIG. 5 as repeated for a plurality of medical context items, additional independent factors may be identified through further analysis of the data. For example, some patient groups, such as those defined by demographic or other information, may be low-efficacy by one or more of the applied protocols. By sorting collected information from the evaluation of protocols, low-efficacy patient groups may be identified. Further analysis of protocols for such patient groups may be warranted.

As illustrated in FIG. 6, the disclosed techniques for evaluating a plurality of patient protocols associated with a medical context include accessing, with a computer system, such as computer system 10, and a database, such as data storage system 12, medical information for a plurality of patients associated with the medical context items (120). For each of the plurality of patients, the medical information includes an indication one of the plurality of patient protocols is associated with the patient.

Computer system 10 further evaluates each of the plurality of patient protocols based on medical information associated with patients within a patient population, the patient population represents a subset of the plurality of patients, to estimate an efficacy of each of the plurality of patient protocols for the patient population (122). For example, the patient population may be defined according to patient demographic information, medical facility information, medical condition details, or by other information. In addition to the numerous other examples disclosed herein, the patient population may be defined according to one or more of: a chief complaint of a patient, a history of present illness of the patient, a past medical history of the patient, a social history of the patient, a family history of the patient, a review of systems of the patient, allergies of the patient, medications of the patient, impressions of the patient by a clinician, a medical plan for the patient, diagnostic imaging results performed on the patient, results of a medical test of the patient, a gender of the patient, an ethnicity of the patient, an age of the patient, a physical attribute of the patient, physical signs of the patient, and physical systems of the patient.

Upon reviewing one or more patient population subsets, computer system 10 may identify one or more of the patient populations as representing a low-efficacy patient population based on the efficacy estimates for the patient population (124). For example, computer system 10 may determine that each of the plurality of patient protocols for a specific patient population is ineffective based on a comparison of the efficacy estimate for each of the plurality of patient protocols for the patient population. As one particular example, computer system 10 may determine that each of the plurality of patient protocols for a specific patient population is ineffective based on the lack of variation in the outcomes of the protocols. Such lack of variation may indicate each of the evaluated protocols is ineffective. For example, among a variety of patient reminders associated with a medical context, if none of the reminders are effective at changing patient behavior for a specific patient population, there will be a lack of variation at predictive outcomes for the medical context among the specific patient population. Computer system 10 may further identify the patient population as a low-efficacy patient population comprises finding a lack of compliance with the plurality of patient protocols for the patient population based on the medial information for the patients of the patient population.

Computer system 10 may store an indication the patient population represents the low-efficacy patient population within data storage system 12 (126).

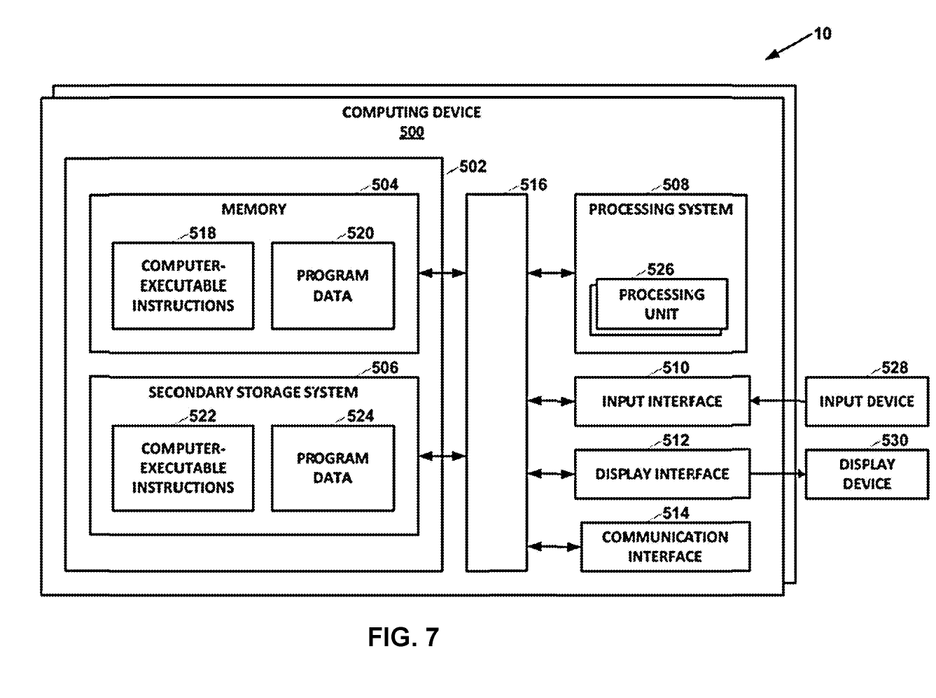

FIG. 7 is a block diagram of an example configuration of a computer system 10, which may be used to preform techniques disclosed herein, including the techniques of FIGS. 2-6. For example, computer system 10 may be used to evaluate a plurality of patient protocols associated with a medical context. In the example of FIG. 7, computer system 10 comprises a computing device 500 and one or more other components.

Computing device 500 is a physical device that processes information. In the example of FIG. 7, computing device 500 comprises a data storage system 502, a memory 504, a secondary storage system 506, a processing system 508, an input interface 510, a display interface 512, a communication interface 514, and one or more communication media 516. Communication media 516 enables data communication between processing system 508, input interface 510, display interface 512, communication interface 514, memory 504, and secondary storage system 506. Computing device 500 can include components in addition to those shown in the example of FIG. 7. Furthermore, some computing devices do not include all of the components shown in the example of FIG. 7.

A computer system-readable medium may be a medium from which a processing system can read data. Computer system-readable media may include computer system storage media and communications media. Computer system storage media may include physical devices that store data for subsequent retrieval. Computer system storage media are not transitory. For instance, computer system storage media do not exclusively comprise propagated signals. Computer system storage media may include volatile storage media and non-volatile storage media. Example types of computer system storage media may include random-access memory (RAM) units, read-only memory (ROM) devices, solid state memory devices, optical discs (e.g., compact discs, DVDs, Blu-ray discs, etc.), magnetic disk drives, electrically-erasable programmable read-only memory (EEPROM), programmable read-only memory (PROM), magnetic tape drives, magnetic disks, and other types of devices that store data for subsequent retrieval. Communication media may include media over which one device can communicate data to another device. Example types of communication media may include communication networks, communications cables, wireless communication links, communication buses, and other media over which one device is able to communicate data to another device.

Data storage system 502 may be a system that stores data for subsequent retrieval. In the example of FIG. 7, data storage system 502 comprises memory 504 and secondary storage system 506. Memory 504 and secondary storage system 506 may store data for later retrieval. In the example of FIG. 7, memory 504 stores computer system-executable instructions 518 and program data 520. Secondary storage system 506 stores computer system-executable instructions 522 and program data 524. Physically, memory 504 and secondary storage system 506 may each comprise one or more computer system storage media.

Processing system 508 is coupled to data storage system 502. Processing system 508 may read computer system-executable instructions from data storage system 502 and executes the computer system-executable instructions. Execution of the computer system-executable instructions by processing system 508 may configure and/or cause computing device 500 to perform the actions indicated by the computer system-executable instructions. For example, execution of the computer system-executable instructions by processing system 508 can configure and/or cause computing device 500 to provide Basic Input/Output Systems (BIOS), operating systems, system programs, application programs, or can configure and/or cause computing device 500 to provide other functionality.

Processing system 508 may read the computer system-executable instructions from one or more computer system-readable media. For example, processing system 508 may read and execute computer system-executable instructions 518 and 522 stored on memory 504 and secondary storage system 506.

Processing system 508 may comprise one or more processing units 526. Processing units 526 may comprise physical devices that execute computer system-executable instructions. Processing units 526 may comprise various types of physical devices that execute computer system-executable instructions. For example, one or more of processing units 526 may comprise a microprocessor, a processing core within a microprocessor, a digital signal processor, a graphics-processing unit, or another type of physical device that executes computer system-executable instructions.

Input interface 510 may enable computing device 500 to receive input from an input device 528. Input device 528 may comprise a device that receives input from a user. Input device 528 may comprise various types of devices that receive input from users. For example, input device 528 may comprise a keyboard, a touch screen, a mouse, a microphone, a keypad, a joystick, a brain-computer system interface device, or another type of device that receives input from a user. In some examples, input device 528 is integrated into a housing of computing device 500. In other examples, input device 528 is outside a housing of computing device 500. In some examples, input device 528 may receive indications of independent and dependent variables from a user and/or other types of data as described above for evaluation of a plurality of patient protocols associated with a medical context.

Display interface 512 may enable computing device 500 to display output on a display device 530. Display device 530 may be a device that presents output. Example types of display devices include printers, monitors, touch screens, display screens, televisions, and other types of devices that display output. In some examples, display device 530 is integrated into a housing of computing device 500. In other examples, display device 530 is outside a housing of computing device 500. In some examples, display device 530 may present evaluation summaries of a plurality of protocols to a user.

Communication interface 514 may enable computing device 500 to send and receive data over one or more communication media. Communication interface 514 may comprise various types of devices. For example, communication interface 514 may comprise a Network Interface Card (NIC), a wireless network adapter, a Universal Serial Bus (USB) port, or another type of device that enables computing device 500 to send and receive data over one or more communication media. In some examples, communication interface 514 may receive medical documents, indications of medical contexts, protocols associated with the medical contexts and/or other types of data as described above. Furthermore, in some examples, communication interface 514 may output evaluation results for a plurality of patient protocols associated with a medical context and/or other types of data as described above.

### EXAMPLES

### Example 1—Protocol Selection, Context Identification, and Performance Metric Definition

Protocols are created and selected based upon the context of their use. One protocol may be assigned for a particular context or multiple protocols may be defined for a particular context. As previously described, a medical context is defined as a circumstance that forms the setting of a medical related event, procedure, diagnoses, or statement that needs improving and/or evaluation. In the area of empirical research, the context would be an independent variable. There are a plurality of methods to specifying the context(s) for a given protocol and/or patient. It may be manually identified through human interactions such as pull-down menus on a computer application to automatically assigning the context by analyzing the medical documents associated with the patient's encounter. For example, an assigned International Classification of Diseases (ICD) code automatically generated from a document using NLP. As an example, an ICD-10-CM coded electronic health record (EHR) would return 2015/16 ICD-10-CM E11.9 to define “Type 2 diabetes mellitus without complications.” The ‘E11.9’ code along with the certainty of discharge would define the context for the “diabetes discharge protocol.” Patients with other diagnosed conditions (e.g., disease, a broken bone, deep vein thrombosis, etc.) would provide different contexts and thus separate protocols may be assigned and used.

Instructions may be provided to a patient that identify steps that should be accomplished to improve the patient's outcome and the quality of care. They may be contained in a textual document, such as a checklist. For a patient being discharged with newly diagnosed diabetes, instructions may include, for example: 1) scheduling an appointment with a primary care physician, 2) enrolling in an outpatient diabetes educational course, and/or 3) engaging in a discussion with a dietician. In some instances, instructions may have not been documented and are verbally transferred from facility staff members as best practices.

Facilities identify and assign protocols for ensuring that the provided instructions are followed and that appropriate feedback was acquired. Protocols may be captured in textual documents (e.g., checklists) or, in many instances, may not have been documented. Typically, they are verbally conveyed and transferred to and from facility personnel.

Examples of protocols for diabetes discharge include scheduling automated patient reminders, which may include text messages, emails, automated telephone calls, personal telephone calls, electronic calendar appointment, and/or wearable device alerts. For the purposes of this example, the protocols for diabetes discharge are defined as 1) provide the patient with the checklist, 2) provide the checklist and recommend a follow-up phone call from the facility to ensure that the appointment was made and the patient understood how to manage their diabetes, e.g., checklist and call, or 3) provide the patient with the checklist and make the appointment for the patient and schedule transportation to pick up the patient, e.g., checklist and transport. These protocols may be converted into digital form and uploaded into a database, library, or repository to be accessed for further processing when required. Such protocols may be hand written and then electronically scanned or may be created electronically through word processing methods and stored in the database. Each context creates one or more protocols and each would be stored in the database. The set of all protocols stored in the database is referred to as P. For many protocols there may be a need for coordination between different aspects of the facility. A protocol may require that a follow-up call be made to the patient in certain number of days (e.g., seven) or that transportation is scheduled to pick up the patient from home and take them to their primary care physician. Once the protocol is selected, computer system 10 may automatically place the call or put the item on the queue to be done manually by an individual at the facility. In some examples, computer system 10 might automatically place the request to have transportation arrive at the patient's home at a particular time and day to bring them to the follow-up appointment.

In addition to having the protocols, context may also be identified. Sets of context are referred to as C. As previously defined, the context is simply the situation that needs improving and/or evaluation. A particular context is referenced as C_{Name} so the context for diabetes discharge is referred to as C_{Diabetes}. A specific protocol for a particular context is specified as: Protocol_{Context}^{N} where “Context” is a label of the context and N is the N^{th} protocol for that context. For example, the first protocol for diabetes discharge is defined as: Protocol_{Diabetes}¹. The notation presupposes that the protocols for a particular context (e.g., diabetes) are evaluated against one another.

Along with generating protocols and identifying the context computer system 10 also needs the ability to evaluate the performance of the protocols. There may be more than one performance metric. For example, in the context of diabetes discharge, the performance metrics include: 1) whether or not there was a re-admission, 2) the patient's weight (and/or change in weight), 3) blood-glucose levels (and/or change in blood-glucose levels), 4) whether the patient filled their medication, and/or 5) whether the patient attended a scheduled appointment. Machine learning algorithms use these performance metrics to evaluate the effectiveness of the above-mentioned protocols. When there is a single metric, computer system 10 can easily evaluate the performance by looking at that metric. However, when there is more than one metric, computer system 10 has to create a utility function that combines these values into a single value. One approach is to do this is to take a weighted sum of the individual metrics where each metric (m) has a weight (w). The utility function may typically consist of multiple possible metrics (M) that are combined to give a single utility value. A simple way to do this is to put an individual weight (W) on each of the metrics and sum the weighted metrics, e.g., U=sum(W*M).

For a particular context, the user may specify the metrics that may be measured for the patient. The metrics may be manually selected through a menu interface, obtained by a survey or questionnaire, or may be automatically proposed based upon previously analyzed context and protocol assessment. Many of the metrics may simply be part of the data being collected about the patient (e.g., the patient's weight, re-admissions) and may be captured with the medical documentation (e.g., EHR). Other data may be specific to a particular context (e.g., blood-glucose levels) and may also be stored/documented in the EHR.

### Example 2—Protocol Assignment

The assignment of protocols to individual patients may be accomplished using any suitable machine-learning algorithms. Such suitable machine learning algorithms include, reinforcement learning (e.g., ε-Greedy, Greedy, Softmax), active learning and other approaches. Machine learning systems would utilize the set of actions (e.g., protocols) (P), the patient's current context (C), and the utility function (U) to evaluate and assign protocols to specific patients. One approach is to implement reinforcement learning that uses ε-Greedy to balance gathering new performance data (e.g., explore) with acquired information and knowledge (e.g., exploit) to understand the impact of a particular action. Randomly selecting protocols, without reference to probability distributions often lead to poor assignment and evaluation performance.

While many such algorithms may be suitable, for the purpose of a working example, two methods are discussed below: Greedy and ε-Greedy.

The Greedy method chooses the preferred protocol based upon computer systems' 10 estimate of the outcomes given a defined performance metric (M). As an example, if a fifty year male is diagnosed as newly diabetic and the objective function (e.g., utility) is to reduce the possibility of readmission, then the greedy method would select the dietician protocol because it has the lowest expected re-admission value (e.g., <5%).

A disadvantage of the Greedy method may be in “exploiting” the previous knowledge. It rarely provides any “exploration” of whether the dynamical system is changing or whether more data would generate a different outcome. The c-Greedy algorithm selects the best protocol 1-E of the time and randomly selects one of the other protocols c of the time. If c is set to 0.1 computer system 10 would generate a random value between 0 and 1. If the value was between 0 and 0.9, the method would select the best protocol (again “dietician”) but if the value is between 0.9 and 1.0 it would randomly select one of the other protocols and assign and record the assignment of the protocol to the patient. As mentioned, other machine learning algorithms could also be used.

One problem in machine learning techniques is a “cold start.” Without information about the expected outcome it is difficult to start a machine learning process. To overcome this problem, a uniform value may be assigned to all of the actions (e.g., protocols), e.g., in the absence of information to distinguish the protocols. A “Greedy” state system may randomly choose between the options with equal probability to being the analysis in this example. As data arrive, computer system 10 may begin to learn the expected outcomes and naturally adjust its assignments based upon the expected outcomes. Another way to begin a cold start is too apply a guess or intuition about the outcomes prior to starting a machine learning algorithm. In this case, a user may preset values for one or more actions according to those intuitions. Again, computer system 10 may automatically adjust those estimates as actions are assigned and metrics are received. The third way is to use prior literature or existing data within the facility to set these expected outcomes. Adding knowledge to computer system 10 may allow computer system 10 to learn faster, e.g., determine the preferred action faster than without any knowledge prior to starting a machine learning algorithm.

Table 1 provides an example data set of 10 patients with a context of newly diagnosed diabetic patient population for protocol assignment. This table provides an example of information that would be available for the protocol evaluation service. This example includes information about the patient's demographic (e.g., age) and the assigned protocol and for 8 of the 10 patients this example includes information on whether the patient was re-admitted or not. Other demographic information of the patient might be available, such as sex, zip code, ethnicity, social support, payment method, and in addition to re-admission, other object-type variables may be tracked if available (such as weight, blood sugar, etc.)

In this example, the random method was used in the ε-Greedy method where ε is set to 0.2. This means that 20% of patients may be assigned randomly. Elder is defined as an age greater than or equal to 60, Middle is classified as greater than or equal to 30, but less than 60, and Young is defined as less than 30 years old.

TABLE 1

| | | | Random | | | Re- |
|---|---|---|---|---|---|---|
| | Age | Class | Value | Method | Protocol | admitted |
| Patient 1 | 25 | Young | 0.9635 | Random | Dietician | No |
| Patient 2 | 35 | Middle | 0.5904 | Greedy | Fitness | No |
| | | | | | Monitor | |
| Patient 3 | 45 | Middle | 0.5983 | Greedy | Fitness | No |
| | | | | | Monitor | |
| Patient 4 | 55 | Middle | 0.8171 | Random | Dietician | Yes |
| Patient 5 | 65 | Middle | 0.8677 | Random | Dietician | No |
| Patient 6 | 50 | Middle | 0.1299 | Greedy | Fitness | No |
| | | | | | Monitor | |
| Patient 7 | 70 | Elder | 0.7716 | Greedy | Home Visit | No |
| Patient 8 | 45 | Middle | 0.4324 | Greedy | Home Visit | Yes |
| Patient 9 | 40 | Middle | 0.3050 | Greedy | Fitness | NA |
| | | | | | Monitor | |
| Patient 10 | 20 | Young | 0.1319 | Greedy | Fitness | NA |
| | | | | | Monitor | |

Patient 1, as defined in Table 1, was 25 year olds and exceeded the randomly selected E value (e.g., 0.9635>0.8) and therefore a protocol was randomly selected and the patient was assigned the dietician protocol. Instructions are provided to the patient at discharge with the ‘dietician’ protocol describing that the patient participate in three phone calls with a dietician to discuss nutrition and diet. After collecting information regarding communication preferences and eating habits, an email is sent from the dietician to the patient with a recommend grocery list and six meal options. After following the protocol, the patient was not re-admitted to a facility for diabetic related incidents in the next thirty days.

Patient 2 was 35 year olds and did not exceed the randomly selected E value (e.g., 0.5904<0.8) and therefore a protocol was greedily selected and the patient was assigned the fitness monitor protocol. Instructions are provided to the patient at discharge with the ‘fitness monitor’ protocol describing that the patient self-monitor diet, exercise, and general health. After automatically collecting information regarding communication preferences, diet, and exercise, data on the fitness monitor would be downloaded and sent to personnel at the facility for evaluation. After following the protocol, the patient was not re-admitted to a facility for diabetic related incidents in the next thirty days.

In the current example we keep the epsilon value at a constant value. However, one can modify this value by decreasing the epsilon value relative to the confidence in the estimate of the outcome. For example, with real values (such as change in weight) when the variance around the value is high (meaning that there is high volatility) one can increase epsilon. This would force computer system 10 to do more “exploration” (e.g., generate more samples for the condition even when it is not the best performing protocol). Adding more samples may decrease the variance and thus decrease the need for computer system 10 to explore.

Eventually, the system may use additional information beyond the medical context definition to further refine the predictive outcomes. For example, the system might know the patient's demographic information (e.g., age, gender, social support system, etc.). As the system collects more performance information it can begin to leverage these factors to further refine the predictive outcomes associated with a medical context item, and select a protocol. Using statistical techniques such as linear regression, random forest, logistic regression these factors can be added to the assignment step.

Using these factors that are outside of the medical context allows the system to start assigning protocols based upon more individualized aspects. For example, given 2 protocols, it is possible that overall, Protocol-A is better than Protocol-B. However, as more data is collected it might be that Protocol-B is better for Male patients who are newly diagnosed diabetics and Protocol-A is better for females.

The techniques described in this disclosure may be implemented, at least in part, in hardware, software, firmware, or any combination thereof. For example, various aspects of the described techniques may be implemented in a wide variety of computer devices, such as servers, laptop computers, desktop computers, notebook computers, tablet computers, hand-held computers, smart phones, and the like. Any components, modules or units have been described provided to emphasize functional aspects and does not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

Within such examples and others, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

The techniques described in this disclosure may also be embodied or encoded in a computer system-readable medium, such as a computer system-readable storage medium, containing instructions. Instructions embedded or encoded in a computer system-readable medium, including a computer system-readable storage medium, may cause one or more programmable processors, or other processors, to implement one or more of the techniques described herein, such as when instructions included or encoded in the computer system-readable medium are executed by the one or more processors. Computer system readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer system readable media. In some examples, an article of manufacture may comprise one or more computer system-readable storage media.

Various examples have been described. These and other examples are within the scope of the following claims.

What is claimed is:

1. A computer-implemented method of evaluating a plurality of protocols associated with a medical context, the method comprising:
receiving, with a computer system, an indication of a medical context item corresponding to a medical context;
accessing, with the computer system, a digital library including a plurality of protocols associated with the medical context;
assigning, with the computer system, predictive outcomes to one or more of plurality of protocols;
selecting, with the computer system, one of the plurality of protocols associated with the medical context based upon the assigned predictive outcomes; and
storing, with the computer system within a database, an indication the selected protocol is assigned to the medical context item.

2. The method of claim 1, further comprising:
monitoring, with the computer system, medical information associated with the medical context item following the selection of the selected protocol;
evaluating, with the computer system, the selected protocol based upon the medical information associated with the medical context item according to one or more performance measures to update the predictive outcome for the selected protocol; and
storing, with the computer system, the updated predictive outcome for the selected protocol within the database.

3. The method of any of claim 2, wherein the performance measures includes a characterization of compliance with the selected protocol.

4. The method of claim 1, wherein selecting the one protocol based upon the assigned predictive outcomes is chosen randomly.

5. The method of claim 1, wherein selecting the one protocol is based upon a counter-balanced assignment of medical contexts to protocols.

6. The method of claim 1, wherein selecting the one protocol is based upon maximizing information expected to be obtained by the selection.

7. The method of claim 1, wherein selecting the one protocol comprises selecting the one of the plurality of protocols according to a machine learning algorithm.

8. The method of claim 1, further comprising populating, with the computer system, the digital library with one or more protocols of the plurality of protocols associated with the medical context.

9. The method of claim 1, wherein at least one of the plurality of protocols represents a protocol associated with an institution associated with the medical context item.

10. The method of claim 1, wherein at least one of the plurality of protocols represents a protocol associated with an institution that is not associated with the medical context item.

11. The method of claim 1, wherein at least one or more protocols is based on peer reviewed literature.

12. The method of claim 1, further comprising sending instructions to an institution staff member and/or a patient corresponding to the selected protocols.

13. The method of claim 1, wherein the medical context defines the medical context item according to one or ore more of:
a location of treatment;
a status of the location;
a chief complaint of a patient;
a history of present illness of the patient;
a past medical history of the patient;
a social history of the patient;
a family history of the patient;
a review of systems of the patient;
allergies of the patient;
medications of the patient;
impressions of the patient by a clinician;
a medical plan for the patient;
diagnostic imaging results performed on the patient;
results of a medical test of the patient;
a gender of the patient;
an ethnicity of the patient;
an age of the patient;
a physical attribute of the patient;
physical signs of the patient; and
physical systems of the patient.

14. The method of claim 1, wherein the assigned predictive outcomes include indications of one or ore more of:
patient readmission rates;
medical condition relapse rates;
patient symptoms;
patient physiological metrics;
medical expenses;
length of stay;
patient satisfaction;
patient life expectancy; and
patient quality of life.

15. The method of claim 1, wherein the indication of the medical context item corresponding to the medical context includes a quantitative indication.

16. The method of claim 1, wherein the indication of the medical context item corresponding to the medical context includes a user indication received via a user interface of the computer system.

17. The method of claim 1, further comprising:
monitoring, with the computer system, medical information associated with the medical context item following the selection of the selected protocol;
evaluating, with the computer system, the selected protocol based upon the medical information associated with the medical context item according to one or more performance measures to update a predictive outcome for the selected protocol of a subset of medical context items corresponding to the medical context; and
storing, with the computer system, a predictive outcome for the subset of medical context items and the selected protocol within the database.

18. The method of claim 17, further comprising:
identifying, with the computer system, a low-efficacy patient group based on the predictive outcome for the subset of medical context items and the selected protocol; and
storing, with the computer system, an indications of the low-efficacy patient group within the database.

19. The method of claim 18, wherein the low-efficacy patient group is defined according to one or more of:
a chief complaint of the subject;
a history of present illness of the subject;
a past medical history of the subject;
a social history of the subject;
a family history of the subject;
a review of systems of the subject;
allergies of the subject;
medications of the subject;
impressions of the subject by a clinician;
a medical plan for the subject;
diagnostic imaging results performed on the subject;
results of a medical test of the subject;
a gender of the subject;
an ethnicity of the subject;
an age of the subject;
a physical attribute of the subject;
physical signs of the subject; and
physical systems of the subject.

20. A computer system-readable storage medium that stores computer system-executable instructions that, when executed, configure a computer system to perform the method of claim 1.

21. A computer system comprising one or more processors configured to perform the method of claim 1.
